(19) United States
(12) Patent Application Publication
(10) Pub. No.: US 2010/0029716 A1
(43) Pub. Date: Feb. 4, 2010

(54) NOVEL 1,2,3,4- TETRAHYDROQUINOLINE DERIVATIVES

(75) Inventors: Roger Tung, Lexington, MA (US); Robert Zelle, Lexington, MA (US)

Correspondence Address: EDWARDS ANGELL PALMER & DODGE LLP, P.O. BOX 55874, BOSTON, MA, 02205, US

(73) Assignee: CONCERT PHARMACEUTICALS, INC., Lexington, MA (US)

(21) Appl. No.: 12/296,927

(22) PCT Filed: Apr. 10, 2007
(86) PCT No.: PCT/US07/08781

§ 371 Date: Sep. 30, 2009

Related U.S. Application Data
(60) Provisional application No. 60/791,052, filed on Apr. 10, 2006.

(51) Int. Cl. A61K 31/47; C07D 215/00; G01N 31/00; C12Q 1/00

(52) U.S. Cl. 514/313; 546/159; 436/8; 435/4

(57) ABSTRACT

The present invention relates to a novel derivative of Compound 1 selectively substituted with fluorine and/or deuterium and optionally further substituted with deuterium and ¹³C elsewhere in the molecule. The compounds of this invention are cholesterol esterase transfer protein inhibitors and possess unique biopharmaceutical and pharmacokinetic properties compared to Compound 1. They may also be used to accurately determine the concentration of Compound 1 in biological fluids and tissues and to determine metabolic patterns of Compound 1 and its isotopologues. The invention further provides compositions comprising these compounds and methods of treating diseases and conditions that are responsive to inhibition of cholesterol esterase transfer protein, alone and in combination with additional agents.

### RELATED APPLICATION

This application claims the benefit of U.S. provisional patent application No. 60/791,052, filed Apr. 10, 2006, the contents of which are incorporated herein by reference in their entirety.

### TECHNICAL FIELD OF THE INVENTION

The present invention relates to novel derivatives of Compound 1, chemically described variously as (2R,4S)-ethyl 4-((3,5-bis(trifluoromethyl)benzyl)-(methoxycarbonyl)amino)-2-ethyl-6-(trifluoromethyl)-3,4-dihydroquinoline-1(2H)-carboxylate, and as (2R,4S)-4-[N-[3,5-Bis(trifluoromethyl)benzyl]-N-(methoxycarbonyl)amino]-2-ethyl-6-(trifluoromethyl)-1,2,3,4-tetrahydroquinoline-1-carboxylic acid ethyl ester; and to derivatives, pharmaceutically acceptable salts, solvates, and hydrates thereof. This invention also provides compositions comprising a compound of this invention and the use of such compositions in methods of treating diseases and conditions that are beneficially treated by selective cholesterol esterase transfer protein (CETP) inhibitors, such as atherosclerosis, peripheral vascular disease; blood lipid, cholesterol, triglyceride and protein disorders; cardiovascular disorders, angina, ischemia and ischemic injury, angioplastic restenosis, hypertension, vascular complications of diabetes, obesity and endotoxemia. The invention also provides methods for the use of a compound of this invention to determine concentrations of Compound 1, particularly in biological fluids and tissues, and to determine metabolism patterns of Compound 1 and the compounds of this invention.

### BACKGROUND OF THE INVENTION

High density lipoprotein (HDL) cholesterol comprises a class of plasma lipoprotein particles that are composed of apolipoproteins, enzymes, and cholesterol esters. In contrast with low density lipoprotein (LDL) and very low density lipoprotein (VLDL) cholesterol, which are associated with increased risk of atherosclerosis and cardiovascular disease, high HDL cholesterol levels are epidemiologically associated with decreased risk of cardiovascular morbidity; Gordon D H and Rifkind B M, N. Engl. J. Med. 1989 321: 1311; Assmann G et al. Atherosclerosis 1996; 124 (suppl 6):S11; Genest J J Jr. et al. Circulation 1992 85: 2025. HDL cholesterol has anti-inflammatory effects and is associated with reverse cholesterol transport, removing cholesterol from peripheral tissues, including foam cells in atherosclerotic lesions, and transporting it back to the liver. Treatment of patients suffering from acute coronary syndrome with recombinant human HDL was demonstrated to result in a highly significant reduction in atheroma volume; Nissen S et al. JAMA 2003 290: 2292.

Cholesterol ester transfer protein (CETP) is a plasma glycoprotein that facilitates transfer of cholesterol esters from high density lipoproteins to LDL and VLDL. It thereby plays a key role in determining plasma lipoprotein particle homeostasis. Tall Ark., J. Lipid Res. 1993 34: 1255.

Compound 1, chemically described variously as (2R,4S)-ethyl 4-((3,5-bis(trifluoromethyl)benzyl)-(methoxycarbonyl)amino)-2-ethyl-6-(trifluoromethyl)-3,4-dihydroquinoline-1(2H)-carboxylate, and as (2R,4S)-4-[N-[3,5-Bis(trifluoromethyl)benzyl]-N-(methoxycarbonyl)amino]-2-ethyl-6-(trifluoromethyl)-1,2,3,4-tetrahydroquinoline-1-carboxylic acid ethyl ester, is graphically represented as:

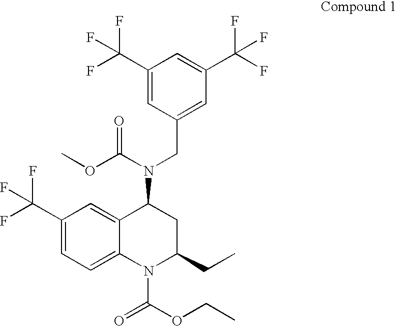

Compound 1, its prodrugs, and pharmaceutically acceptable salts of said compounds and prodrugs, and certain related compounds, are known as potent and selective cholesterol esterase transfer protein (CETP) inhibitors. Compound 1 has been demonstrated to alter blood cholesterol composition, raising high density lipoprotein (HDL) cholesterol levels while concurrently reducing non-HDL cholesterol levels; e.g. see Durley R C et al., J. Med. Chem. 2002 45: 3891; Clark R W et al., Arterioscler. Thromb. Vasc. Biol. 2004 24: 490; Bays H et al., Expert Rev. Cardiovasc. Ther. 2005 3: 789. Compound 1 and related compounds are useful for the treatment of a variety of medical conditions in mammals, such as humans, including atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension; vascular complications of diabetes, obesity or endotoxemia. DeNinno M P et al., U.S. Pat. Nos. 6,197,786; 6,140,343; 6,147,089; 6,147,090; 6,310,075; 6,395,751; 6,489,478; Goldstein S W et al., U.S. Pat. Nos. 6,140,342; 6,147,090; 6,362,198; 6,395,751; DeNinno M P et al., U.S. Patent Application nos. 20040092550 and 20050245570. Each referenced publication, patent, patent application, and internet page is incorporated herein in its entirety by reference. Despite intensive efforts to develop and implement pharmacological and behavioral intervention, arterioscleroses and associated diseases remain the leading cause of mortality in industrialized countries and great need remains for improved therapeutic options for its treatment.

Definitions and descriptions of these conditions are known to the skilled practitioner and are further delineated, for instance, in the above patents and references contained therein. See also Harrison's Principles of Internal Medicine 16th Edition, Kasper D L et al. Eds., 2004, McGraw-Hill Professional; and Robbins & Cotran Pathologic Basis of Disease, Kumar V et al. Eds., 2004, W.B. Saunders.

The combination of CETP inhibitors, in particular Compound 1, with a second agent selected from an HMG-CoA reductase inhibitor, a glucosidase and/or amylase inhibitor, and a growth hormone secretagogue, extends or enhances the utility of Compound 1 to the treatment of familial hyperlipidaemias, obesity, diabetes mellitus, dyslipidaemias, combined hyperlipidaemias, hypercholesterolaemias, hypertriglyceridaemias, primary or secondary prevention of coronary heart disease including myocardial infarction, atherosclerosis, peripheral vascular disease, cardiovascular disorders, angina, ischemia, cardiac ischemia, stroke, reperfusion injury, angioplastic restenosis, hypertension, vascular complications of diabetes, endotoxemia and intermittent claudication; Schmeck C et al., U.S. Pat. No. 5,932,587; DeNinno M P et al., U.S. Pat. No. 6,197,786; Curatolo W J et al., US Patent Application 20050038007.

Compound 1 has been characterized in vitro and ex vivo by determining its ability to inhibit transfer of ³H-labeled cholesteryloleate from exogenous tracer HDL to the non-HDL fraction in human plasma, and from the ³H-labeled LDL to the HDL fraction in plasma of mice possessing transgenic human CETP and apolipoprotein A1 (hCETP mice); DeNinno M P et al., U.S. Pat. No. 6,197,786; Curatolo W J et al., US Patent Application 20050038007; Durley R C et al., J. Med. Chem. 2002 45: 3891.

Transgenic hCETP mice treated with Compound 1 also demonstrated decreased rates of cholesterol oleate transfer from LDL donor particles to HDL, as well as increased HDL cholesterol upon multiple dosing. Compound 1 was also found to reduce the formation of atherosclerotic lesions in a rabbit model of atherosclerosis; Morehouse L A et al., Circulation 2004 110 (17, Suppl. 3): Abst. 1168.

In a clinical trial with healthy volunteers, Compound 1 demonstrated a dose-dependent ability to decrease CETP activity with an EC₅₀ of 43 nM. HDL cholesterol levels were increased by up to 91% during a 14-day dosing period with concomitant reductions of up to 42% LDL cholesterol and stable total cholesterol levels; Clark R W et al., Arterioscler. Throm. Vasc. Biol. 2004 24: 490. Compound 1 also proved effective in raising HDL-cholesterol in patients with a low HDL cholesterol background both alone and in combination with the HMG CoA reductase inhibitor atorvastatin; Brousseau M E et al., N. Engl. J. Med. 2004 350: 1505; Brousseau M E et al., Arteriscler. Throm. Vasc. Biol. 2005 25-1057.

Compound 1 is substantially more effective as a single agent when dosed twice daily versus once daily at 120 mg (Clark R W et al., Arterioscler. Throm. Vasc. Biol. 2004 24: 490). This greater efficacy appears to directly reflect the observed fall-off in CETP inhibition, resulting from falling blood levels of Compound 1. Notwithstanding that fact, the current clinical development of Compound 1 is being undertaken with a once-daily regimen. Compound 1 is quite hydrophobic and possesses poor aqueous solubility, resulting in low oral bioavailability of conventional formulations. This difficulty has been addressed by the development of applicable controlled-release formulations; see Curatolo W J et al., US Patent Application 20030198674. A complementary approach to enhancing the exposure of Compound 1 would be to increase the biological half-life of a Compound 1 derivative, so that a given amount of administered agent would produce a longer-lasting effect. Such an effect could be produced, for instance, by reducing the metabolic rate of the compound. Combinations of the two approaches would be particularly advantageous.

Accordingly, it is desirable to provide a compound that has the beneficial activities of Compound 1 and may also have other benefits, e.g., reduced adverse side effects, with a decreased metabolic liability, to further extend its pharmacological effective life, enhance patient compliance and, potentially, to decrease population pharmacokinetic variability and/or decrease its potential for dangerous drug-drug interactions.

### SUMMARY OF THE INVENTION

The present invention provides a compound of formula 1 or 2:

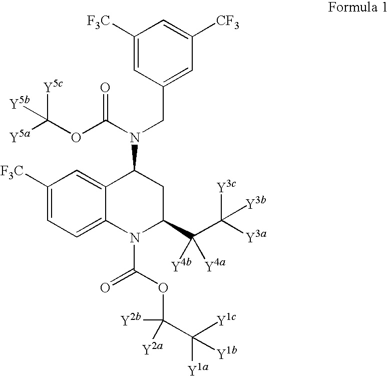

Wherein each Y (including Y^{1a}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c}) is independently selected from the group consisting of hydrogen, deuterium, or fluorine; and wherein at least one Y is deuterium; or a pharmaceutically acceptable salt, solvate, or hydrate thereof.

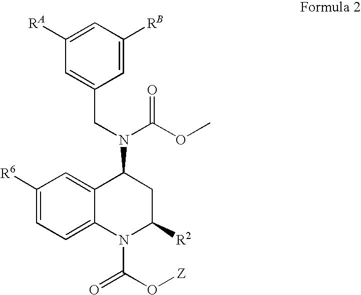

wherein:
R^{A} and R^{B} are each independently trifluoromethyl, deuteromethyl or chloro;
R² is ethyl, isopropyl, cyclopropyl, cyclobutyl, or methoxymethyl;
R⁶ is trifluoromethyl, deuteromethyl or chloro;
Z is ethyl, propyl, isopropyl, tert-butyl, or 2-hydroxyethyl, each optionally substituted with from 1-9 independent substituents, each selected from fluorine and hydroxyl groups;
   wherein one or more H atoms in Z or R² is replaced by deuterium; or a pharmaceutically acceptable salt, solvate, or hydrate thereof.
   Embodiments of compounds of formula 1 or 2 include those wherein:
Y^{1a}, Y^{1b}, and Y^{1c} are each deuterium; or
Y^{1a}, Y^{1b}, Y^{1c}, Y^{2a} and Y^{2b} are each deuterium; and/or
Y^{3a}, Y^{3b}, and Y^{3c} are each deuterium; and/or
Y^{4a}, and Y^{4b} are each deuterium; and/or
Y^{5a}, Y^{5b} and Y^{5c} are each deuterium.

Other embodiments of compounds include those that contain five or more deuterium atoms; or those wherein at least five of Y^{1a}, Y^{1b}, Y^{1c}, Y^{2a}, Y^{2b}, Y^{3a}, Y^{3b}, Y^{3c}, Y^{4a}, Y^{4b}, Y^{5a}, Y^{5b} and Y^{5c} are deuterium.

Additional embodiments of compounds include those that contain six or more deuterium atoms.

The compounds of this invention demonstrate reduced rates of oxidative metabolism due to the replacement of hydrogen by deuterium or fluorine. This results in enhanced pharmacological effects and the potential for reduced dosing of compounds of formula I to achieve similar or superior medical effects as compared to dosing of a similar quantity of Compound 1.

The compounds of this invention and compositions comprising them, are useful to reduce or ameliorate severity, duration, or progression, or enhance function compromised by, a disorder beneficially treated by inhibition of CETP or by increased HDL cholesterol levels. In one embodiment, the invention provides a method of preventing or reducing the severity of a condition selected from atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension, reperfusion injury, vascular complications of diabetes, obesity and endotoxemia, said method comprising the step of administering to a subject suffering from said condition a composition comprising a compound of formula 1 or 2, and a pharmaceutically acceptable carrier.

The compounds and compositions of this invention are also useful as analytical reagents for determining the concentration of Compound 1 in a solution.

### DETAILED DESCRIPTION OF THE INVENTION

In one embodiment, the present invention provides a compound of formula 1 or 2:

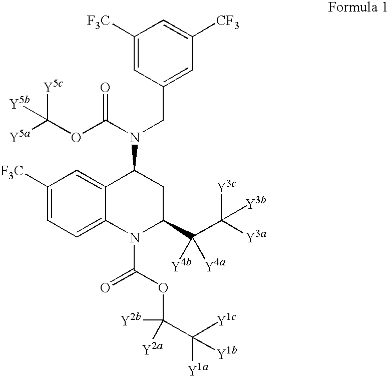

Wherein each Y (including Y^{1c}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c}) is independently selected from the group consisting of hydrogen, deuterium, or fluorine; and wherein at least one Y is deuterium; or a pharmaceutically acceptable salt, solvate, or hydrate thereof.

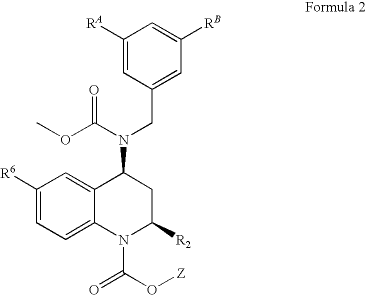

wherein:
R^{A} and R^{B} are each independently trifluoromethyl, deuteromethyl or chloro;
R² is ethyl, isopropyl, cyclopropyl, cyclobutyl, or methoxymethyl;
R⁶ is trifluoromethyl, deuteromethyl or chloro;
Z is ethyl, propyl, isopropyl, tert-butyl, or 2-hydroxyethyl, each optionally substituted with from 1-9 independent substituents, each selected from fluorine and hydroxyl groups;
   wherein one or more H atoms in Z or R² is replaced by deuterium; or a pharmaceutically acceptable salt, solvate, or hydrate thereof.
   Other aspects of the compounds of formula 2 are those wherein R^{A}=R^{B}=trifluoromethyl; R^{A} and R^{B} are the same; wherein one or more H atoms in Z is substituted by deuterium; wherein one or more H atoms in R² is substituted by deuterium; and wherein one or more H atoms in each of Z and R² is substituted by deuterium; and wherein any single carbon atom in formula 2 is di-deuterated; wherein any single carbon atom in formula 2 is tri-deuterated; or wherein any alkyl group is per-deuterated.

Other groups of compounds of formula 1 or 2 include those wherein:
Y^{1a}, Y^{1b}, and Y^{1c} are each deuterium; and/or
Y^{1a}, Y^{1b}, Y^{1c}, Y^{2a} and Y^{2b} are each deuterium; and/or
Y^{3a}, Y^{3b}, and Y^{3c} are each deuterium; and/or
Y^{4a}, and Y^{4b} are each deuterium; and/or
Y^{5a}, Y^{5b}, and Y^{5c} are each deuterium.

Embodiments of compounds include those that contain five or more deuterium atoms.

Embodiments of compounds include those that contain six or more deuterium atoms.

In another embodiments, independently other structural classes of formula 1 or 2 are shown in Table 1. In this table, each open space represents hydrogen present at its natural isotopic abundance. For each of these structural classes, another embodiment is that wherein each carbon is present at its natural isotopic abundance. Other embodiments are members of structural classes in which each carbon is present at its natural abundance, and either all hydrogens not otherwise specified as deuterium are present at their respective natural abundances, or that each of the hydrogens in the methyl group of the methyl carbamate is replaced with deuterium and otherwise all hydrogens not otherwise specified as deuterium are present at their respective natural abundances.

TABLE 1
Exemplary Compound Classes of the Invention

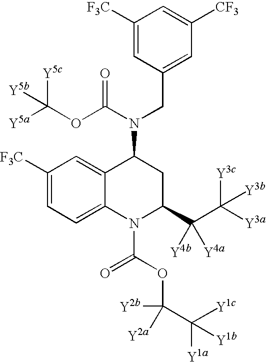

| Compound | | | | | | | | | | | | | |
|---|---|---|---|---|---|---|---|---|---|---|---|---|---|
| Class | Y^{1a} | Y^{1b} | Y^{1c} | Y^{2a} | Y^{2b} | Y^{3a} | Y^{3b} | Y^{3c} | Y^{4a} | Y^{4b} | Y^{5a} | Y^{5b} | Y^{5c} |
| 2 | D | D | D | | | | | | | | | | |
| 3 | D | D | D | D | D | | | | | | | | |
| 4 | D | D | D | | | D | D | D | | | | | |
| 5 | D | D | D | D | D | D | D | D | | | | | |
| 6 | | | | | | D | D | D | D | D | | | |
| 7 | D | D | D | | | D | D | D | D | D | | | |
| 8 | D | D | D | D | D | D | D | D | D | D | | | |
| 9 | | | | | | D | D | D | | | | | |
| 10 | | | | | | | | | | | D | D | D |
| 11 | D | D | D | D | D | | | | | | D | D | D |
| 12 | D | D | D | | | D | D | D | | | D | D | D |
| 13 | D | D | D | D | D | D | D | D | | | D | D | D |
| 14 | D | D | D | D | D | D | D | D | D | D | D | D | D |

In each of compound classes 2 through 14 in Table 1, one embodiment is that wherein all hydrogens not otherwise specified as deuterium are present at their respective natural abundances. Other compounds are those wherein each of the hydrogens in the methyl of the methyl carbamate group are replaced with deuterium, and otherwise all hydrogens not otherwise specified as deuterium are present at their respective natural abundances.

In yet another embodiment, the compound is selected from any one of the compounds set forth in Table 2 (below):

TABLE 2
Exemplary Embodiments of Formula I

| Compound | Y^{1a} | Y^{1b} | Y^{1c} | Y^{2a} | Y^{2b} | Y^{3a} | Y^{3b} | Y^{3c} | Y^{4a} | Y^{4b} | Y^{5a} | Y^{5b} | Y^{5c} |
|---|---|---|---|---|---|---|---|---|---|---|---|---|---|
| 101 | D | D | D | H | H | H | H | H | H | H | H | H | H |
| 102 | D | D | D | D | D | H | H | H | H | H | H | H | H |
| 103 | D | D | D | H | H | D | D | D | H | H | H | H | H |
| 104 | D | D | D | D | D | D | D | D | H | H | H | H | H |
| 105 | H | H | H | H | H | D | D | D | D | D | H | H | H |
| 106 | D | D | D | H | H | D | D | D | D | D | H | H | H |
| 107 | D | D | D | D | D | D | D | D | D | D | H | H | H |
| 108 | H | H | H | H | H | D | D | D | H | H | H | H | H |
| 109 | H | H | H | H | H | H | H | H | H | H | D | D | D |
| 110 | D | D | D | D | D | H | H | H | H | H | D | D | D |
| 111 | D | D | D | H | H | D | D | D | H | H | D | D | D |
| 112 | D | D | D | D | D | D | D | D | H | H | D | D | D |
| 113 | D | D | D | D | D | D | D | D | D | D | D | D | D |

It is understood that the beneficial effect of selective substitution of deuterium for naturally abundant hydrogen can also be applied similarly to structurally related analogs of Compound 1, for instance those disclosed in DeNinno M P et al., U.S. Pat. Nos. 6,197,786; 6,140,343; 6,147,089; 6,147,090; 6,310,075; 6,395,751; 6,489,478; Goldstein S W et al., U.S. Pat. Nos. 6,140,342; 6,147,090; 6,362,198; 6,395,751; DeNinno M P et al., US Patent Application nos. 20040092550 and 20050245570.

The terms “compound of formula I”, “compound of formula 2” and “compounds of this invention” as used herein, are intended to include any salts, solvates, and hydrates of said compound.

Throughout this specification, reference to “each Y” includes, independently, all “Y” groups (Y^{1a}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c}), where applicable.

In the compounds of this invention any atom not specifically designated as a particular isotope is meant to represent any stable isotope of that atom unless otherwise stated. Unless otherwise stated, when a position is designated specifically as “H” or “hydrogen”, the position is understood to have hydrogen at its natural abundance isotopic composition.

It will be recognized that some variation of natural isotopic abundance occurs in a synthesized compound depending upon the origin of chemical materials used in the synthesis. Thus, a preparation of a particular compound will inherently contain small amounts of deuterated and/or ¹³C-containing isotopologues. The concentration of such naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66: 15; Ganes L Z et al., Comp. Biochem. Physiol. A Mol. Integr. Physiol. 1998, 119: 725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom.

The term “deuteromethyl” as used herein refers to a methyl group having 1-3 deuterium atoms, e.g., CH₂D, CHD₂, or CD₃. A preferred deuteromethyl is CD₃.

The term “isotopologue” refers to a species that differs from a specific compound of this invention only in the isotopic composition thereof.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio.

As used herein, the term “hydrate” means a compound which further includes a stoichiometric or non-stoichiometric amount of water bound by non-covalent intermolecular forces.

The term “solvate” means a compound which further includes a stoichiometric or non-stoichiometric amount of solvent such as water, acetone, ethanol, methanol, dichloromethane, 2-propanol, or the like, bound by non-covalent intermolecular forces.

As used herein, the term “polymorph” means solid crystalline forms of a compound or complex thereof. Different polymorphs of the same compound can exhibit different physical, chemical and/or spectroscopic properties. Different physical properties include, but are not limited to stability (e.g., to heat, light or moisture), compressibility and density (important in formulation and product manufacturing), hygroscopicity, solubility, and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

Another aspect of the invention is a compound of this invention for use in the treatment or prevention in a subject (e.g., a human) of a disease, disorder or symptom thereof delineated herein.

Another aspect of the invention is the use of a compound of the invention in the manufacture of a medicament for treatment or prevention in a subject (e.g., a human) of a disease, disorder or symptom thereof delineated herein.

The compounds of the invention may be synthesized by well-known techniques. The starting materials and certain intermediates used in the synthesis of the compounds of this invention are available from commercial sources or may themselves be synthesized using reagents and techniques known in the art, including those synthesis schemes delineated herein. See, for instance, Durley R C et al., J. Med. Chem. 2002 45: 3891; McIntyre J A and Castaner J, Drugs Fut. 2005 30: 344; Goldstein S W et al., U.S. Pat. No. 6,140,342 to Pfizer; DeNinno M P et al., U.S. Pat. No. 6,197,786; and Damon D B et al., U.S. Pat. No. 6,313,142. Each of these documents is incorporated in its entirety herein by reference.

Such methods can be carried out utilizing corresponding deuterated and optionally, other isotope-containing reagents to synthesize the compounds and intermediates delineated herein, or invoking standard synthetic protocols known in the art for introducing isotopic atoms to a chemical structure. Intermediates can be used with or without purification (e.g., filtration, distillation, sublimation, crystallization, trituration, solid phase extraction, and chromatography).

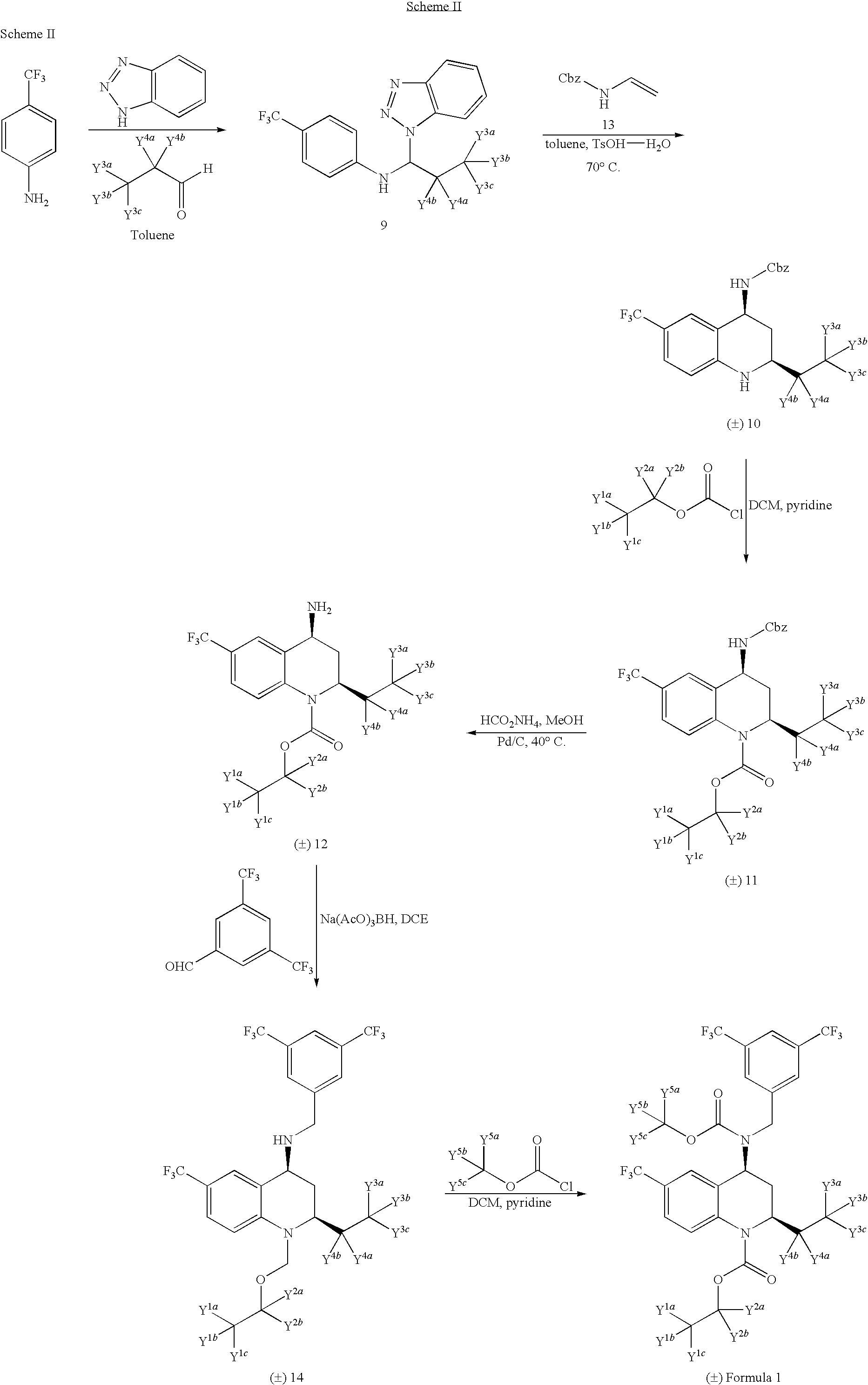

One convenient method for producing a compound of formula I is graphically illustrated in Scheme II, wherein each Y is independently hydrogen, fluorine, or deuterium and wherein at least one Y in each of formulae 1, 11, 12 and 14 is deuterium. As shown in Scheme II, a nitrogen atom can be protected with a suitable nitrogen protecting group (PG) such as Cbz (as shown), 4-methoxy-Cbz, Fmoc, Boc, Alloc, or other group known in the art. A nitrogen atom may also be protected with bivalent protection, such as N,N-dibenzyl or phthalyl. In this scheme, certain intermediates and final products such as formulae 10, 11, 12, 14 and 1, can exist as resolved enantiomers or as enantiomeric mixtures. Such mixtures can be separated by, for example, chiral HPLC methods or converting the enantiomeric mixture into a diastereomeric mixture by reaction with an appropriate optically active compound (e.g., a chiral alcohol), separating the diastereomers and converting (e.g., hydrolyzing) the individual diastereomers to the corresponding pure enantiomers. Also, an enantiomeric mixture which contains a basic moiety may be separated into its compounding pure enantiomers by forming a diastereomeric salt with an optically pure chiral acid (e.g., tartaric acid or camphorsulfonic acid) and separating the diastereomers by fractional crystallization followed by neutralization to break the salt, thus providing the corresponding pure enantiomers. All such isomers, including diastereomers, enantiomers and mixtures thereof are considered as part of this invention.

Formula I compounds of this invention may be obtained in enantiomerically enriched form by resolving the racemate of the final compound or an intermediate in its synthesis (preferably the final compound) employing chromatography (preferably high pressure liquid chromatography [HPLC]) on an asymmetric resin (such as Chiralcel™ AD or OD [obtained from Chiral Technologies, Exton, Pa.]) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

In Scheme II, condensation of propionaldehyde, or a fluorine or deuterium-substituted derivative thereof, with 4-trifluoromethylaniline and benzotriazole, is conducted to yield a compound of formula 9. Cycloaddition of an N-protected vinylamine (13) then produces a 1,2,3,4-tetrahydroquinoline of formula 10. The Cbz-protected vinylamine (compound 13) can be prepared as shown in Scheme III (below) according to the method of Jessup, P J et. al., Organic Syntheses, Coll. Vol. 6, 1988, p. 95. Acylation of the secondary amine with ethyl chloroformate, or a fluorine or deuterium-substituted derivative thereof, yields a compound of formula 11, which is subjected to N-deprotection to unmask the primary amine. Resolution of this amine by fractional crystallization; for instance, using (−)-2,3-bis(benzoyloxy)succinic acid (derived from L-tartaric acid) or (−) 2,3-bis(4-methylbenzoyloxy)succinic acid (derived from L-tartaric acid) as the counterion; or chiral chromatography by means known in the art, may then be conducted to produce the resolved amine of formula 12. This compound is reductively alkylated with 3,5-bis(trifluoromethyl)benzaldehyde and finally acylated with methyl chloroformate or, in certain instances with trideuteromethyl chloroformate, yielding the product of formula 1.

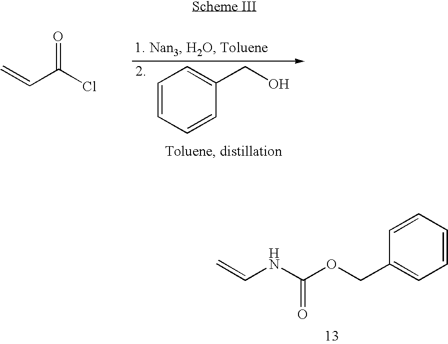

Variations in reactants, reaction conditions, and synthetic approaches are described the above-cited synthetic references, and others will be evident to those of

Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artisan by use of available structure-searchable database software, for instance, SciFinder® (CAS division of the American Chemical Society) and CrossFire Beilstein® (Elsevier MDL), or internet search engines such as Google® or keyword databases such as the US Patent and Trademark Office text database.

The synthetic methods described herein may additionally include steps, either before or after any of the steps described in Scheme II, to add or remove suitable protecting groups in order to ultimately allow synthesis of a particular compound of formula 1 or 2.

According to another embodiment, the invention provides any of the above-described intermediate compounds of formulae 9, 10, 11, and 12, in which each Y is independently selected from hydrogen, deuterium and fluorine, and wherein at least one Y is deuterium.

Combinations of substituents and variables envisioned by this invention are only those that result in the formation of stable compounds. The term “stable”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to CETP inhibition).

The term “isotopologue” refers to species that differ from a specific compound of this invention only in the isotopic composition of their molecules or ions.

A specific compound of this invention may also be referred to as a “heavy atom isotopic compound” to distinguish it from its lighter isotopologues when discussing mixtures of isotopologues.

Chemical naming terminology can be complex and different chemical names can often reasonably be applied to the same structure. To avoid any confusion, “Compound 1” refers to the chemical structure shown herein for that compound.

It will be recognized that many commonly occurring atoms in biological systems exist naturally as mixtures of isotopes. Thus, any macroscopic amount of Compound 1, although designated in its formula as being devoid of deuterium and ¹³C, when synthesized inherently contains small amounts of deuterated and ¹³C-containing isotopologues. The present invention excludes such minor amounts of said isotopologues (“variant isotopologues”) from its scope in that the term “compound” as used in this invention refers to a composition of matter that is predominantly the specific carbon and hydrogen isotopologue designated by its formula. A compound, as defined herein, in embodiments contains less than 10%, less than 6%, and less than 3% of all other carbon and hydrogen isotopologues, including Compound 1, as variant isotopologues. Compositions of matter that contain greater than 10% of all other specific carbon and hydrogen isotopologues combined are referred to herein as mixtures and must meet the parameters set forth below. These limits of isotopic composition, and all references to isotopic composition herein, refer solely to the carbons and hydrogens of the compound of Formula I and do not include the isotopic composition of other atom types, for instance solvent entrapped as a solvate or excipients used in formulating compounds of this invention.

The term “heavy atom” refers to isotopes of higher atomic weight than the predominant naturally occurring isotope.

The term “stable heavy atom” refers to non-radioactive heavy atoms.

Both “²H” and “D” refer to deuterium.

“Stereoisomer” refers to both enantiomers and diastereomers

“NMR” refers to nuclear magnetic resonance spectroscopy

“PDE” refers to cyclic guanosine monophosphate-specific phosphodiesterase

“cGMP” in the context of a chemical agent refers to cyclic guanosine monophosphate

“5′-GMP” refers to guanosine-5′-monophosphate

“cAMP” refers to cyclic adenosine monophosphate

“5′-AMP” refers to adenosine-5′-monophosphate

“Antagonist” refers to both antagonists and inverse agonists

“PM” refers to poor metabolizer

“EM” refers to extensive metabolizer

“AIBN” refers to 2,2′-azo-bis(isobutyronitrile)

“Alloc” refers to allyloxycarbonyl

“Boc” refers to tert-butoxycarbonyl

“Cbz” refers to benzyloxycarbonyl or carbobenzyloxy

“Fmoc” refers to 9-fluorenylmethoxycarbonyl

“MeOH” refers to methanol

“EtOH” refers to ethanol

“AcOH” and “HOAc” both refer to acetic acid

“THF” refers to tetrahydrofuran

“DMF” refers to N,N-dimethylformamide

“aq.” refers to aqueous

“h” refers to hours

“min” refers to minutes

“brine” refers to saturated aqueous sodium chloride

“US” refers to the United States of America

“FDA” refers to Food and Drug Administration

“IND” refers to Investigational New Drug

“NDA” refers to New Drug Application

“cGMP” in the context of synthesis or manufacturing of drug substance or drug product refers to current Good Manufacturing Practices

“CAS” refers to the chemical abstracts service of the American Chemical Society

“AUC” refers to area under the plasma-time concentration curve

CYP3A4 refers to cytochrome P450 oxidase isoform 3A4

“MC-4R” refers to the human melanocortin-4 receptor

“5-HT” refers to 5-hydroxytryptamine or serotonin

“NEP” refers to neutral endopeptidease (EC 3.4.24.11)

“HMG-CoA” refers to 3-hydroxy-3-methylglutaryl-coenzyme A

“ETA” refers to endothelin subtype A receptors

“ETB” refers to endothelin subtype B receptors

“PPAR” refers to peroxisome proliferator-activated receptor

“CETP” refers to cholesterol esterase transfer protein

Both “patient” and “subject” used in the context of methods of treatment according to this invention refer to a mammal, preferably an economically important species such as pets and livestock (e.g., dog, cat, bird, chicken, turkey, horse, cow, steer, lamb, sheep), and more preferably a human.

The invention further provides compositions comprising a mixture of a compound of this invention and its lighter isotopologues. These mixtures may occur, for instance, simply as the result of an inefficiency of incorporating an isotope at a given position; intentional or inadvertent exchange of protons for deuterium, e.g. exchange of bulk solvent for heteroatom-attached deuterium; or intentional mixtures of pure compounds.

In one embodiment, such mixtures comprise at least about 50% of the heavy atom isotopic compound (i.e., less than about 50% of lighter isotopologues). Another aspect is a mixture comprising at least 80% of the heavy atom isotopic compound. Another aspect is a mixture comprising 90% of the heavy atom isotopic compound.

In an alternate embodiments the mixture comprises a compound of Formula I and its lighter isotopologues in relative proportions such that at least about 50%, at least 80%, at least 90%, at least 95% or at least 98% of the compounds in said mixture comprise a stable heavy atom isotope at each position designated as a stable heavy atom isotope in the chemical formula of the heavy atom isotopic compound.

The invention also provides compositions comprising an effective amount of a compound of formula 1 or 2 (or any formulae herein) and an acceptable carrier. Another aspect is, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington: The Science And Practice Of Pharmacy, University of the Sciences in Philadelphia (Ed.); Lippincott Williams & Wilkins (21st ed. 2005).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Poorly water-soluble drug substances can benefit through formulation as amorphous molecular dispersions or powders, which improve their wetability and dissolution kinetics; e.g. see Singhal D and Curatolo W, Adv. Drug Deliv. Rev. 2004 56: 335 and references therein. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued and pending U.S. patents, some of which include, but are not limited to, U.S. Pat. Nos. 4,369,172, 4,842,866 and 6,706,283, and references cited therein. Coatings can be used for delivery of compounds to the intestine (see, e.g., U.S. Pat. Nos. 6,638,534; 5,217,720; and 6,569,457; 6,461,631; 6,528,080; 6,800,663; and references cited therein).

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. Surfactants such as sodium laurylsulfate may be useful to enhance dissolution and absorption.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as ethanol.

The pharmaceutical compositions of this invention may be administered in the form of suppositories for rectal or vaginal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition will be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetylesters wax, cetearylalcohol, 2-octyldodecanol, benzylalcohol and water. The pharmaceutical compositions of this invention may also be topically applied to the lower intestinal tract by rectal suppository formulation or in a suitable enema formulation. Topically-transdermal patches and iontophoretic administration are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzylalcohol or other suitable preservatives, absorption promoters to enhance bioavailability, fluorocarbons, and/or other solubilizing or dispersing agents known in the art.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to another embodiment, a compound of this invention may be incorporated into compositions for coating an implantable medical device, such as prostheses, artificial valves, vascular grafts, stents, or catheters. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinylacetate, and mixtures thereof. The coatings are optionally further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating or filling an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released form said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

The present invention further provides pharmaceutical compositions comprising an effective amount of one or more compound of the invention in combination with an effective amount of a second therapeutic agent useful for treating or preventing a condition selected from familial hyperlipidaemias, obesity, diabetes mellitus, dyslipidaemias, combined hyperlipidaemias, hypercholesterolaemias, hypertriglyceridaemias, primary or secondary prevention of coronary heart disease including myocardial infarction, atherosclerosis, peripheral vascular disease, cardiovascular disorders, angina, ischemia, cardiac ischemia, stroke, reperfusion injury, angioplastic restenosis, hypertension, vascular complications of diabetes, endotoxemia and intermittent claudication.

Such second therapeutic agents useful in combination with the compounds of this invention include, but are not limited to: an HMG-CoA reductase inhibitor, a glucosidase inhibitor and/or amylase inhibitor, and a growth hormone secretagogue; pharmaceutically acceptable salts, solvates, hydrates, and polymorphs of the foregoing; and combinations thereof.

Examples of HMG-CoA reductase inhibitors include, but are not limited to, atorvastatin, rosuvastatin, cerivastatin, fluvastatin, lovastatin, pravastatin, simvastatin, itavastatin, dalvastatin, NK-104-LH, crilvastatin, bervastatin, glenvastatin, and BMY-21950.

Examples of glucosidase and/or amylase inhibitors include, but are not limited to, acarbose, adiposine, voglibose, miglitol, emiglitate, MDL-25637, camiglibose (MDL-73945), tendamistate, AI-3688, trestatin, pradimicin-Q and salbostatin.

Examples of growth hormone secretagogues include, but are not limited to, MK-0677, L-162752 and L-163022; NN703 and ipamorelin; hexarelin; GPA-748 (KP102, GHRP-2); and LY444711.

In another embodiment, the invention provides separate dosage forms of a compound of this invention and a second therapeutic agent, wherein said compound and said second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, a compound of the present invention is present in an effective amount. As used herein, the term “effective amount” refers to an amount which, when administered in a proper dosing regimen, is sufficient to reduce or ameliorate the severity, duration or progression, or enhance function compromised by a disorder responsive to inhibition of CETP; to cause the regression of a disorder associated with the transfer of cholesterol esters from HDL to LDL and VLDL; or to enhance or improve the prophylactic or therapeutic effect(s) of another therapy.

In certain preferred embodiments, treatment according to the invention provides a reduction in or prevention of at least one symptom or manifestation of a disorder that has been linked to CETP function, as determined by in vivo or in vitro inhibition of at least about 10%, alternatively 20%, 30%, 40%, 50%, 60%, 70%, 80%, 90%, 95%, 98% or 99% of such activity. With respect to inhibition of the CETP term “effective amount” means an amount that results in one or more of: reduction of cholesterol ester transfer from high density lipoproteins to LDL and VLDL; the correction of or relief from a behavior, deficit, symptom, syndrome or disease, or enhancement of otherwise compromised function that has been linked to CETP function or that is known to be responsive to the inhibition of CETP activity, alone or in combination with another agent or agents; or the induction of a behavior, activity or response that has been linked to inhibition of CETP activity.

The interrelationship of dosages for animals and humans (based on milligrams per meter squared of body surface) is described in Freireich et al., (1966) Cancer Chemother. Rep. 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 500 mg/kg, more preferably 0.01 mg/kg to about 50 mg/kg, yet more preferably 0.025 mg/kg to about 1.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise second therapeutic agents, an effective amount of the other agent is between about 20% and 100% of the dosage normally utilized in a monotherapy regime using just that additional agent. Preferably, an effective amount is between about 70% and 100% of the normal monotherapeutic dose. The normal monotherapeutic dosages of these second therapeutic agents are well known in the art. See, e.g., Wells B G et al., eds., Pharmacotherapy Handbook, 6th Edition, McGraw-Hill Medical. (2005); Fleming T ed., PDR Pharmacopoeia Pocket Dosing Guide 2006, Thomsom (2005); 2006 Physicians' Desk Reference, Thomson P D R; Package edition (2005) each of which references are entirely incorporated herein by reference.

It is expected that some of the second therapeutic agents listed above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In one embodiment, the present invention provides a method of causing CETP inhibition in a subject, comprising the step of administering to said subject an effective amount of a compound of this invention, preferably as part of a composition additionally comprising a pharmaceutically acceptable carrier. Preferably, this method is employed to treat a mammalian subject suffering from or susceptible to one or more disease or disorder selected from atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension, vascular complications of diabetes, obesity, or endotoxemia. Other embodiments include any of the methods herein wherein the subject is identified as in need of the indicated treatment.

In another embodiment, the method of treatment further comprises the step of administering to said patient a second therapeutic agent which is effective to treat coronary heart disease including myocardial infarction, atherosclerosis, peripheral vascular disease, cardiovascular disorders, angina, ischemia, cardiac ischemia, stroke, reperfusion injury, angioplastic restenosis, hypertension, diabetes, including its vascular complications, endotoxemia, and intermittent claudication.

In each of the above embodiments, the second therapeutic agent may be administered together with a compound of this invention as part of a single dosage form or as separate dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administering of the second therapeutic agent may occur before, concurrently with, and/or after the administering of the compound of this invention. When administration of the second therapeutic agent occurs concurrently with a compound of this invention, the two (or more) agents may be administered in a single dosage form (such as a composition of this invention comprising a compound of the invention and an second therapeutic agent as described above), or in separate dosage forms. The administration of a composition of this invention comprising both a compound of the invention and a second therapeutic agent to a subject does not preclude the separate administration of said second therapeutic agent, any other therapeutic agent or any compound of this invention to said subject at another time during a course of treatment.

Effective amounts of a second therapeutic agent useful in the methods of this invention are well known to those skilled in the art and guidance for dosing may be found in patents referenced herein. However, it is well within the skilled artisan's purview to determine the second therapeutic agent's optimal effective-amount range. In one embodiment of the invention where a second therapeutic agent is administered to an animal, the effective amount of the compound of this invention is less than its effective amount would be where the second therapeutic agent is not administered. In another embodiment, the effective amount of the second therapeutic agent is less than its effective amount would be where the compound of this invention is not administered (i.e., the amount of second therapeutic agent administered in a monotherapy). In this way, undesired side effects associated with high doses of either agent may be minimized. Other potential advantages (including without limitation improved dosing regimens and/or reduced drug cost) will be apparent to those of skill in the art.

Second therapeutic agents useful in the methods of treatment of this invention are the same as those described above as part of combination compositions.

The compounds of this invention may be assayed for activity in vitro by known methods. For instance, kits allowing the measurement of CETP activity are commercially available (Roar Biomedical, Inc., New York, N.Y.). Methodology for such assays is also well known; see for instance Bisgaier C L et al., J. Lipid Res. 1993 34: 1625; Lagrost L et al. Clin. Chem. 1995 41: 914; Schmeck C et al., U.S. Pat. No. 5,932,587; DeNinno M P et al., U.S. Pat. No. 6,197,786.

Animal models to assess the ability of CETP inhibitors to transfer cholesterol esters in vivo are also known in the art. Hamsters and rabbits are suitable models wherein inhibiting CETP results in elevation of HDL cholesterol; see e.g. Evans, G. F., et al: J. Lipid Res. 1994 35: 1634; and Whitlock, M. E., et al: J. Clin. Invest. 1989 84: 129. Transgenic mice expressing human apolipoprotein B and human CETP are also valuable since they measure effects of test compounds on the human cholesterol transfer system; see e.g. Grass D S et al., J. Lipid Res. 1995 36: 1082. Transgenic mice expressing this combination of proteins are commercially available through Taconic, Hudson, N.Y. Each of the compounds of this invention may be tested by such means.

### Diagnostic Methods and Kits

The compounds and compositions of this invention are also useful as reagents in methods for determining the concentration of Compound 1 in solution or biological sample such as plasma, examining the metabolism of Compound 1 and other analytical studies.

According to another embodiment, the invention provides a method of determining the concentration of Compound 1 in a biological sample, said method comprising the steps of:

a) adding a known concentration of a compound of Formula 1, or a salt, solvate, or hydrate thereof to said biological sample;

b) subjecting said biological sample to a measuring device that distinguishes Compound 1 from said compound of Formula 1;

c) calibrating said measuring device to correlate the detected quantity of Compound 1, respectively, with the known concentration of said compound of Formula 1, respectively, added to said biological sample; and

d) determining the concentration of said compound in said biological sample by comparing the detected quantity of Compound 1 with the detected quantity and known concentration of said compound of Formula 1.

Measuring devices that can distinguish Compound 1 from said compound of Formula 1 include any measuring device that can distinguish between two compounds that are of identical structure except that one contains one or more heavy atom isotope versus the other. In one embodiment, such a measuring device is a mass spectrometer.

In certain embodiments, the compound of Formula 1 includes at least three deuterium atoms.

In another embodiment, the compound of Formula 1 includes at least four deuterium atoms.

In another embodiment, the compound of Formula 1 includes at least five deuterium atoms.

In another embodiment, the compound of Formula 1 includes at least six deuterium atoms.

In another embodiment, the method comprises the additional step of organically extracting both Compound 1 and said compound of Formula 1. Compound 1 and the compound of Formula 1 will have similar solubility, extraction, and chromatographic properties, but significantly different molecular mass. Thus, the second compound is useful as an internal standard in a method that comprises the step of organic extraction to measure the efficiency of that extraction and to ensure an accurate determination of the true concentration of Compound 1 (see Tuchman M and McCann M T, Clin. Chem. 1999 45: 571; Leis H J et al., J. Mass Spectrom. 2001 36: 923; Taylor R L et al. Clin. Chem. 200248: 1511).

Compounds of Formula 1 are particularly useful in this method since they are not radioactive and therefore do not pose a hazard to personnel handling the compounds. Thus, these methods do not require precautions beyond those normally applied in clinical sample analysis.

In another embodiment, the invention provides a diagnostic kit comprising:

a) a compound of Formula 1, or a salt, solvate, or hydrate thereof; and

b) instructions for using said compound of Formula 1 to determine the concentration of a test compound in a biological sample.

Stably labeled isotopes have long been used to assist in research into the enzymatic mechanism of cytochrome P450 enzymes (Korzekwa K R et al., Drug Metab. Rev. 1995 27: 45 and references therein; Kraus, J A and Guengerich, F P, J. Biol. Chem. 2005 280: 19496; Mitchell K H et al., Proc. Natl. Acad. Sci. USA 2003 109: 3784).

In another embodiment, the invention provides a method of evaluating the metabolic stability of a compound of Formula 1 or 2, comprising the steps of contacting the compound of Formula 1 or 2 with a metabolizing enzyme source for a period of time; and comparing the amount of said compound and metabolic products of said compounds after said period of time.

In one embodiment, the method comprises an additional step of comparing the amount of said compound and said metabolic products of said compounds at an interval during said period of time. This method allows the determination of a rate of metabolism of said compound.

In another embodiment, the method of evaluating the metabolic stability of a compound of Formula 1 comprises the additional steps of: contacting Compound 1 with said metabolizing enzyme source; comparing the amount of Compound 1 and its metabolic products after said period of time; and comparing the metabolic stability of Compound 1 and said compound of Formula 1. This method is useful in determining whether and at which sites on a compound of Formula 1 additional deuterium substitution may cause increases in metabolic stability. It is also useful in comparing the metabolic stability of a compound of Formula 1 with the metabolic stability of Compound 1.

A metabolizing enzyme source may be a purified, isolated or partially purified metabolic protein, such as a cytochrome P450; a biological fraction, such as a liver microsome fraction; or a piece of a metabolizing organ, such as a liver slice.

The determination of the amount of compound and its metabolic products is well known in the art. It is typically achieved by removing an aliquot from the reaction mixture and subjecting it to an analysis capable of distinguishing between the compound and its metabolites, such as reversed-phase HPLC with UV absorption or mass spectroscopic detection. Concentrations of both the metabolizing enzyme and the compound may be varied to determine kinetic parameters, for instance, by using appropriate nonlinear regression software such as is known in the art. By comparing the kinetic parameters of both a compound of Formula 1 and Compound 1, an apparent steady-state deuterium isotope effect (^{D}(V/K)) can be determined as the ratio of products formed in the hydrogen versus deuterium reactions.

The determination of a rate of metabolism of a compound of Formula 1 may be achieved in a reaction separate from the reaction for determining the metabolism rate of Compound 1. Alternatively, Compound 1 may be admixed, respectively, with a compound of Formula 1 in a competition experiment to determine rates of disappearance of the two compounds, making use of analytical instrumentation capable of differentiating between the two compounds based on their mass differences.

In yet another embodiment, pre-steady state kinetics, such as V₀, may be determined by means known in the art, for instance, using quench-flow apparatus, by monitoring the quenched reactions at varying times after mixing the compound or isotopologue with the metabolizing enzyme source.

In a related embodiment, the invention provides a kit comprising, in separate vessels: a) a compound of any of the formulae herein; and b) a metabolizing enzyme source. The kit is useful for comparing the metabolic stability of a compound of Formula 1 with Compound 1, as well as evaluating the effect of deuterium replacement at various positions on a compound of Formula 1. In another embodiment, the kit further comprises instructions for using a compound of any of the formulae herein and said metabolizing enzyme source to evaluate the metabolic stability of, respectively, a compound of Formula 1 or 2.

Other embodiments are those wherein the compound of Formula 1 is replaced by a compound of any of the formulae delineated herein.

In order that the invention might be more fully understood, the following examples are set forth. They are not intended to limit the scope of the invention and further examples will be evident to those of ordinary skill in the art. In each example set forth herein, carbon shall be ¹²C, and hydrogen shall by ¹H, each incorporated at its natural abundance, unless otherwise specified. All solvents used in the Examples are anhydrous unless otherwise specified.

### Example 1

N-(1-(1H-benzo[d][1,2,3]triazol-1-yl)-propyl)-4-(trifluoromethyl)aniline (Compound 9 wherein all Y═H). A 1000 ml flask under N₂ was charged with benzotriazole (18.5 g, 0.155 mol, 1 eq.) and anhydrous toluene (200 ml). A solution of 4-amino benzotrifluoride (25 g, 0.155 mol, 1 eq.) in toluene (30 ml) was added over 1 min. A solution of propionaldehyde (12.4 ml, 0.17 mol, 1.1 eq.) and toluene (30 ml) was added over 20 min. The reaction was allowed to stir overnight. n-Heptane (200 ml) was added and the slurry stirred for 1 h. The precipitate was filtered to give the title compound: 37.8 g, 76% yield.

### Example 2

N-(1-(1H-benzo[d][1,2,3]triazol-1-yl)-3,3,3-trideuteropropyl)-4-(trifluoromethyl)aniline (Compound 9 wherein Y3a-c=D). The product was prepared using 3,3,3-trideuteropropionaldehyde (example 57) according to the procedure in example 1 in 79% yield and a purity of >95% by ¹H NMR.

### Example 3

N-(1-(1H-benzo[d][1,2,3]triazol-1-yl)-2,2,3,3,3-pentadeuteoropropyl)-4-(trifluoromethyl)aniline (Compound 9 wherein Y3a-c and Y4a-b=D). The product was prepared using d5-propionaldehyde according to the procedure in example 1 in a 74% yield.

### Example 4

cis-Benzyl-2-ethyl-6-(trifluoromethyl)-1,2,3,4-tetrahydroquinolin-4-ylcarbamate (Compound (±)10 wherein all Y═H). The product from Example 1 (5 g, 15.6 mmol, 1 eq.), N-vinyl-carbamic acid benzylester (example 53; 2.76 g, 15.6 mmol, 1 eq.), pTSA.H₂O (30 mg, 0.156 mmol, 0.01 eq.) were added to a flask containing anhydrous toluene (50 ml). The mixture was heated to 70° C. and stirred for 2 h under an atmosphere of N₂. The reaction was cooled to room temperature. EtOAc (50 ml) was added and the mixture transferred to a separatory funnel. The mixture was washed with 1M NaOH (20 ml), H₂O (20 ml), brine (20 ml) and dried over MgSO₄. The mixture was concentrated in vacuo. The crude mixture was taken up in toluene (50 ml) and heptane (50 ml) was added. The mixture was stirred for 1 h resulting in the formation of a white precipitate. The precipitate was isolated by filtration to give the title compound: 3.8 g, 64% yield.

### Example 5

cis-Benzyl-2-(2,2,2-trideuteroethyl)-6-(trifluoromethyl)-1,2,3,4-tetrahydroquinolin-4-ylcarbamate (Compound (±)10 wherein Y3a-c=D) The product from example 2 was reacted according to the procedure in Example 4 to provide the title compound in 70% yield and a purity of >95% by ¹H NMR.

### Example 6

cis-Benzyl-2-(pentadeuteroethyl)-6-(trifluoromethyl)-1,2,3,4-tetrahydroquinolin-4-ylcarbamate (Compound (±) 0 wherein Y3a-c and Y4a-b=D). The product from example 3 was reacted according to the procedure in Example 4 to provide the title compound in 86% yield with a purity of 94.9% as indicated by LC-MS.

### Example 7

cis-ethyl 4-(benzyloxycarbonylamino)-2-ethyl-6-(trifluoromethyl)-3,4-dihydroquinoline-1(2H)-carboxylate (Compound (±)11 wherein all Y═H). A flask was charged with the product from example 4 (3.8 g, 10 mmol, 1 equiv.), DCM (30 ml) and anhydrous pyridine (4 ml, 50 mmol, 5 equiv.). A solution of ethylchloroformate (4.8 ml, 50 mmol, 5 equiv.) in DCM (10 ml) was added slowly. After complete addition of the chloroformate the reaction was stirred for 1 h. The reaction was cooled to 0-5° C. in an ice bath and 1M NaOH (50 ml) added. The mixture was stirred for 15 min and then transferred to a separatory funnel. The layers were separated and the aqueous extracted with DCM (50 ml). The combined DCM layers were washed with 1M HCl (50 ml), sat. aq. NaHCO₃ solution (50 ml), brine (50 ml) and dried over MgSO₄. The organic mixture was concentrated in vacuo to give the title product, 4.8 g, 106% yield.

### Example 8

cis-ethyl 4-(benzyloxycarbonylamino)-2-(2,2,2-trideuteroethyl)-6-(trifluoromethyl)-3,4-dihydroquinoline-1(2H)-carboxylate (Compound (±)11 wherein Y3a-c=D). The product from example 5 was reacted according to the procedure in Example 7 to provide the title product in 70% yield.

### Example 9

cis-ethyl 4-(benzyloxycarbonylamino)-2-pentadeuteroethyl-6-(trifluoromethyl)-3,4-dihydroquinoline-1(2H)-carboxylate (Compound (±)11 wherein Y3a-c and Y4a-b=D). The product from example 6 was reacted according to the procedure in Example 7 to provide the title compound in 71% yield with a purity of >95% by ¹H NMR.

### Example 10

cis-2,2,2-trideuteroethyl 4-(benzyloxycarbonylamino)-2-ethyl-6-(trifluoromethyl)-3,4-dihydroquinoline-1(2H)-carboxylate (Compound (±)11 wherein Y1a-c=D). The product from example 4 was reacted with the product from example 54 according to the procedure in Example 7 to provide the title compound in 63% yield.

### Example 11

cis-pentadeuteroethyl 4-(benzyloxycarbonylamino)-2-ethyl-6-(trifluoromethyl)-3,4-dihydroquinoline-1(2H)-carboxylate (Compound (±)11 wherein Y1a-c and Y2a-b=D). The product from example 4 was reacted with the product from example 55 according to the procedure in Example 7 to provide the title compound in 73% yield.

### Example 12

cis-2,2,2-trideuteroethyl 4-(benzyloxycarbonylamino)-2-(2,2,2-trideutero)-ethyl-6-(trifluoromethyl)-3,4-dihydroquinoline-1 (2H)-carboxylate (Compound (±)11 wherein Y1a-c and Y3a-c=D). The product from example 5 was reacted with the product from example 54 according to the procedure in Example 7 to provide the title compound in 77% yield with purity >95% by ¹H NMR.

### Example 13

cis-2,2,2-trideuteroethyl 4-(benzyloxycarbonylamino)-2-pentadeuteroethyl-6-(trifluoromethyl)-3,4-dihydroquinoline-1(2H)-carboxylate (Compound (±)11 wherein Y1a-c; Y3a-c; and Y4a-b=D). The product from example 6 was reacted with the product from example 54 according to the procedure in Example 7 to provide the title compound in 64% yield.

### Example 14

cis-pentadeuteroethyl 4-(benzyloxycarbonylamino)-2-(2,2,2-trideutero)-ethyl-6-(trifluoromethyl)-3,4-dihydroquinoline-1 (2H)-carboxylate (Compound (±)11 wherein Y1a-c; Y2a-b; and Y3a-c=D). The product from example 5 was reacted with the product from example 55 according to the procedure in Example 7 to provide the title compound in 77% yield with a purity >95% by ¹H NMR

### Example 15

cis-pentadeuteroethyl 4-(benzyloxycarbonylamino)-2-pentadeuteroethyl-6-(trifluoromethyl)-3,4-dihydroquinoline-1(2H)-carboxylate (Compound (±)11 wherein Y1a-c; Y2a-b; Y3a-c; and Y4a-b=D). The product from example 6 was reacted with the product from example 55 according to the procedure in Example 7 to provide the title compound in 72% yield.

### Example 16

cis-ethyl 4-amino-2-ethyl-6-(trifluoromethyl)-3,4-dihydroquinoline-1(2H)-carboxylate (Compound (±)12 wherein all Y═H). The product from Example 7 (4.52 g, 10.0 mmol, 1 eq.) was added to a 3 necked flask containing MeOH (45 ml). 10% Pd/C (50% wet, 0.45 g) was added and the mixture purged with H₂. The mixture was heated to 40° C. and stirred for 2 h at which point the reaction was complete. The reaction was allowed to cool to room temperature and filtered through a pad of celite. The filtrate was concentrated in vacuo to give the desired product, 3.1 g, 98% yield.

### Example 17

cis-ethyl 4-amino-2-(2,2,2-trideuteroethyl)-6-(trifluoromethyl)-3,4-dihydroquinoline-1(2H)-carboxylate (Compound (±)12 wherein Y3a-c=D). The product from example 8 was reacted according to the procedure in Example 16 to provide the title compound in 86% yield and with a purity of >95% by ¹H NMR

### Example 18

cis-ethyl 4-amino-2-pentadeuteroethyl-6-(trifluoromethyl)-3,4-dihydroquinoline-1(2H)-carboxylate (Compound (±)12 wherein Y3a-c and Y4a-b=D). The product from example 9 was reacted according to the procedure in Example 16 to provide the title compound in 93% yield and a purity of >95% by ¹H NMR.

### Example 19

cis-2,2,2-trideuteroethyl 4-amino-2-ethyl-6-(trifluoromethyl)-3,4-dihydroquinoline-1(2H)-carboxylate (Compound (±)12 wherein Y1a-c=D). The product from example 10 was reacted according to the procedure in Example 16 to provide the title compound in 98% yield.

### Example 20

cis-pentadeuteroethyl 4-amino-2-ethyl-6-(trifluoromethyl)-3,4-dihydroquinoline-1(2H)-carboxylate (Compound (±)12 wherein Y1a-c and Y2a-b=D) The product from example 11 was reacted according to the procedure in Example 16 to provide the title compound in 94% yield.

### Example 21

cis-2,2,2-trideuteroethyl 4-amino-2-(2,2,2-trideutero)-ethyl-6-(trifluoromethyl)-3,4-dihydroquinoline-1(2H)-carboxylate (Compound (±)12 wherein Y1a-c and Y3a-c=D) The product from example 12 was reacted according to the procedure in Example 16 to provide the title compound in 86% yield and with a purity of >95% by ¹H NMR.

### Example 22

cis-2,2,2-trideuteroethyl 4-amino-2-pentadeuteroethyl-6-(trifluoromethyl)-3,4-dihydroquinoline-1(2H)-carboxylate (Compound (±)12 wherein Y1a-c; Y3a-c; and Y4a-b=D). The product from example 13 was reacted according to the procedure in Example 16 to provide the title compound in 92% yield.

### Example 23

cis-pentadeuteroethyl 4-amino-(2,2,2-trideutero)-ethyl-6-(trifluoromethyl)-3,4-dihydroquinoline-1(2H)-carboxylate (Compound (±) 12 wherein Y1a-c; Y2a-b; and Y3a-c=D) The product from example 14 was reacted according to the procedure in Example 16 to provide the title compound in 97% yield and with a purity of >95% by ¹H NMR.

### Example 24

cis-pentadeuteroethyl 4-amino-2 pentadeuteroethyl-6-(trifluoromethyl)-3,4-dihydroquinoline-1(2H)-carboxylate (Compound (±) 12 wherein Y1a-c; Y2a-b; Y3a-c; and Y4a-b=D) The product from example 15 was reacted according to the procedure in Example 16 to provide the title compound in 89% yield.

### Example 25

(2S,4S)-ethyl 4-amino-2-(2,2,2-trideuteroethyl)-6-(trifluoromethyl)-3,4-dihydroquinoline-1(2H)-carboxylate, hemi-(−)-dibenzoyl-L-tartrate salt. Dissolve 2.2 mmol of the product of Example 17 in about 8.5 mL of warm 97% aqueous ethanol. Add 2.2 mmol of (−) dibenzoyl-L-tartaric acid. Cool, seeding with crystals of the title salt. Let stand at room temperature for 17 h and filter, washing with a small volume of cold 97% aqueous ethanol. Dry in vacuo to yield the title compound.

Following a similar procedure, the following Examples are prepared:

### Example 26

(2S,4S)-ethyl 4-amino-2-pentadeuteroethyl-6-(trifluoromethyl)-3,4-dihydroquinoline-1(2H)-carboxylate, hemi-(−)-dibenzoyl-L-tartrate salt is prepared from the product of Example 18.

### Example 27

(2S,4S)-2,2,2-trideuteroethyl 4-amino-2-ethyl-6-(trifluoromethyl)-3,4-dihydroquinoline-1(2H)-carboxylate, hemi-(−)-dibenzoyl-L-tartrate salt is prepared from the product of Example 19.

### Example 28

(2S,4S)-pentadeuteroethyl 4-amino-2-ethyl-6-(trifluoromethyl)-3,4-dihydroquinoline-1(2H)-carboxylate, hemi-(−)-dibenzoyl-L-tartrate salt is prepared from the product of Example 20.

### Example 29

(2S,4S)-2,2,2-trideuteroethyl 4-amino-2-(2,2,2-trideutero)-ethyl-6-(trifluoromethyl)-3,4-dihydroquinoline-1(2H)-carboxylate, hemi-(−)-dibenzoyl-L-tartrate salt is prepared from the product of Example 21.

### Example 30

(2S,4S)-2,2,2-trideuteroethyl 4-amino-2-pentadeuteroethyl-6-(trifluoromethyl)-3,4-dihydroquinoline-1(2H)-carboxylate, hemi-(−)-dibenzoyl-L-tartrate salt is prepared from the product of Example 22.

### Example 31

(2S,4S)-pentadeuteroethyl 4-amino-2-(2,2,2-trideutero)-6-(trifluoromethyl)-3,4-dihydroquinoline-1(2H)-carboxylate, hemi-(−)-dibenzoyl-L-tartrate salt is prepared from the product of Example 23.

### Example 32

(2S,4S)-pentadeuteroethyl 4-amino-2 pentadeuteroethyl-6-(trifluoromethyl)-3,4-dihydroquinoline-1(2H)-carboxylate, hemi-(−)-dibenzoyl-L-tartrate salt is prepared from the product of Example 24.

### Example 33

cis-ethyl 4-(3,5-bis(trifluoromethyl)benzylamino)-2-(2,2,2-trideuteroethyl)-6-(trifluoromethyl)-3,4-dihydroquinoline-1(2H)-carboxylate (Compound (±)₁₋₄ wherein all Y═H). The product from Example 16 (3.1 g, 9.8 mmol, 1 eq.) was added to a flask containing DCE (40 ml). 3,5-Bis(trifluoromethyl)benzaldehyde (1.7 ml, 10.3 mmol, 1.05 eq.) was added and the mixture stirred for 1 h. Na(AcO)₃BH (4.24 g, 19.6 mmol, 2 equiv.) was added and left to stir overnight at room temperature. DCE (20 ml) and 1M NaOH (20 ml) was added to the reaction mixture. The layers were separated and the aqueous extracted with DCE (2×20 ml). The combined organics were washed with 1M HCl (20 ml), sat. aq. NaHCO₃ (20 ml), brine (20 ml) and dried over MgSO₄. The mixture was filtered and the filtrate concentrated in vacuo to give the title product, 4.8 g, 69% yield as a white solid.

### Example 34

(2S,4S)-ethyl 4-(3,5-bis(trifluoromethyl)benzylamino)-2-(2,2,2-trideuteroethyl)-6-(trifluoromethyl)-3,4-dihydroquinoline-1(2H)-carboxylate (Compound (2S,3S)-14 wherein Y3a-c=D). Partition 18.6 mmol of the product of Example 25 between ca. 200 mL of methylene chloride and 40 mL of 1 N NaOH. Wash the organic layer with brine, dry over MgSO₄, and concentrate to about 50 mL. Treat this solution with 19.7 mmol of 3,5-bis(trifluoromethyl)benzaldehyde and stir for 1 h under argon at room temperature. Add 37.2 mmol of sodium triacetoxyborohydride and stir for 20 h. Partition the reaction mixture between 40 mL each of methylene chloride and 1 N NaOH. Extract the aqueous layer with methylene chloride, combine the organic layers, and wash them with 50% brine. Dry (MgSO₄) and concentrate in vacuo. Dissolve the residue in 50 mL of methanol, cool in an ice bath under argon, and add 19.7 mmol of p-toluenesulfonic acid monohydrate. After mixing for several minutes, add 200 mL of isopropylether and concentrate to about 25 mL. Treat with an additional 200 mL of isopropylether and cool in an ice bath under argon. Filter and wash the filtrate with several portions of isopropyl ether to yield the title compound.

Following a similar procedure, the following Examples are prepared:

### Example 35

(2S,4S)-ethyl 4-(3,5-bis(trifluoromethyl)benzylamino)-2-pentadeuteroethyl-6-(trifluoromethyl)-3,4-dihydroquinoline-1(2H)-carboxylate (Compound (2S,4S)-14 wherein Y3a-c and Y4a-b=D) is prepared from the product of Example 26.

### Example 36

(2S,4S)-2,2,2-trideuteroethyl 4-(3,5-bis(trifluoromethyl)benzylamino)-2-ethyl-6-(trifluoromethyl)-3,4-dihydroquinoline-1(2H)-carboxylate (Compound (2S,3S)-14 wherein Y3a-c and Y4a-b=D) is prepared from the product of Example 27.

### Example 37

(2S,4S)-pentadeuteroethyl 4-(3,5-bis(trifluoromethyl)benzylamino)-2-ethyl-6-(trifluoromethyl)-3,4-dihydroquinoline-1(2H)-carboxylate (Compound (2S,3S)-14 wherein Y1a-c and Y2a-b=D) is prepared from the product of Example 28.

### Example 38

(2S,4S)-2,2,2-trideuteroethyl 4-(3,5-bis(trifluoromethyl)benzylamino)-2-(2,2,2-trideutero)-ethyl-6-(trifluoromethyl)-3,4-dihydroquinoline-1(2H)-carboxylate (Compound (2S,3S)-14 wherein Y1a-c and Y3a-c=D) is prepared from the product of Example 29.

### Example 39

(2S,4S)-2,2,2-trideuteroethyl 4-(3,5-bis(trifluoromethyl)benzylamino)-2-pentadeuteroethyl-6-(trifluoromethyl)-3,4-dihydroquinoline-1(2H)-carboxylate (Compound (2S,3S)-14 wherein Y1a-c; Y3a-c; and Y4a-b=D) is prepared from the product of Example 30.

### Example 40

(2S,4S)-pentadeuteroethyl 4-(3,5-bis(trifluoromethyl)benzylamino)-2-(2,2,2-trideuteroethyl)-6-(trifluoromethyl)-3,4-dihydroquinoline-1(2H)-carboxylate (Compound (2S,3S)-14 wherein Y1a-c; Y2a-b; and Y3a-c=D) is prepared from the product of Example 31.

### Example 41

(2S,4S)-pentadeuteroethyl 4-(3,5-bis(trifluoromethyl)benzylamino)-2-pentadeuteroethyl-6-(trifluoromethyl)-3,4-dihydroquinoline-1(2H)-carboxylate (Compound (2S,3S)-14 wherein Y1a-c; Y2a-b; Y3a-c; and Y4a-b=D) is prepared from the product of Example 32.

### Example 42

(2S,4S)-ethyl 4-((3,5-bis(trifluoromethyl)benzyl)(methoxycarbonyl)amino)-2-(2,2,2-trideuteroethyl)-6-(trifluoromethyl)-3,4-dihydroquinoline-1(2H)-carboxylate (compound of Formula 1 wherein Y3a-c=D). Dissolve an 11.4 mmol portion of the product of Example 34 in 25 mL of THF. Under an argon atmosphere, add 40 mmol of finely ground Na₂CO₃. With good stirring, add 28.5 mmol of methyl chloroformate dropwise during several minutes. After an additional 19 h, concentrate to about half volume and partition the residue between 60 mL each of ethyl acetate and half saturated brine. Wash the organic layer with 10% KHSO₄, saturated NaHCO₃, and brine, dry over MgSO₄, and concentrate in vacuo. Purify by silica gel chromatography using methylene chloride/ethyl acetate/hexanes eluant to yield the title compound.

Following a similar procedure, the following Examples are prepared:

### Example 43

### (2S,4S)-ethyl 4-((3,5-bis(trifluoromethyl)benzyl)(methoxycarbonyl)amino)-2-pentadeuteroethyl-6-(trifluoromethyl)-3,4-dihydroquinoline-1(2H)-carboxylate (compound of Formula 1 wherein Y3a-c and Y4a-b=D) is prepared from the product of Example 35.

### Example 44

(2S,4S)-2,2,2-trideuteroethyl 4((3,5-bis(trifluoromethyl)benzyl)-(methoxycarbonyl)amino)-2-ethyl-6-(trifluoromethyl)-3,4-dihydroquinoline-1(2H)-carboxylate (compound of Formula 1 wherein 1a-c=D) is prepared from the product of Example 36.

### Example 45

(2S,4S)-pentadeuteroethyl 4-((3,5-bis(trifluoromethyl)benzyl)-(methoxycarbonyl)amino)-2-ethyl-6-(trifluoromethyl)-3,4-dihydroquinoline-1(2H) carboxylate (compound of Formula 1 wherein Y1a-c and Y2a-b=D) is prepared from the product of Example 37.

### Example 46

(2S,4S)-2,2,2-trideuteroethyl 4-((3,5-bis(trifluoromethyl)benzyl)-(methoxycarbonyl)amino)-2-(2,2,2-trideuteroethyl)-6-(trifluoromethyl)-3,4-dihydroquinoline-1(2H)-carboxylate (compound of formula 1 wherein Y1a-c and Y3a-c=D) is prepared from the product of Example 38.

### Example 47

(2S,4S)-2,2,2-trideuteroethyl 4-((3,5-bis(trifluoromethyl)benzyl)(methoxycarbonyl)amino)-2-pentadeuteroethyl-6-(trifluoromethyl)-3,4-dihydroquinoline-1(2H)-carboxylate (compound of Formula 1 wherein Y1a-c; Y3a-c; and Y4a-b=D) is prepared from the product of Example 39.

### Example 48

(2S,4S)-pentadeuteroethyl 4((3,5-bis(trifluoromethyl)benzyl)-(methoxycarbonyl)amino)-2-(2,2,2-trideuteroethyl)-6-(trifluoromethyl)-3,4-dihydroquinoline-1(2H)-carboxylate (compound of formula 1 wherein Y1a-c; Y2a-b; and Y3a-c=D) is prepared from the product of Example 40.

### Example 49

(2S,4S)-pentadeuteroethyl 4-((3,5-bis(trifluoromethyl)benzyl)(methoxycarbonyl)amino)-2 pentadeuteroethyl-6-(trifluoromethyl)-3,4-dihydroquinoline-1(2H)-carboxylate (compound of Formula 1 wherein Y1a-c; Y2a-b; Y3a-c; and Y4a-b=D) is prepared from the product of Example 41.

### Example 50

(2S,4S)-ethyl 4-((3,5-bis(trifluoromethyl)benzyl)((trideuteromethoxy)carbonyl)amino)-2-pentadeuteroethyl)-6-(trifluoromethyl)-3,4-dihydroquinoline-1(2H)-carboxylate (compound of Formula 1 wherein Y3a-c; Y4a-b; and Y5a-c=D) is prepared according to the general method set forth in Example 42, using the product of Example 35 in place of the product of Example 34, and using trideuteromethyl chloroformate in place of methyl chloroformate.

### Example 51

(2S,4S)-pentadeuteroethyl 4-((3,5-bis(trifluoromethyl)benzyl)((trideuteromethoxy)carbonyl)amino)-2-ethyl)-6-(trifluoromethyl)-3,4-dihydroquinoline-1(2H)-carboxylate (compound of Formula 1 wherein Y1a-c; Y2a-b; and Y5a-c=D) is prepared according to the general method set forth in Example 45, starting from the product of Example 37.

### Example 52

(2S,4S)-2,2,2-trideuteroethyl 4-((3,5-bis(trifluoromethyl)benzyl)((trideuteromethoxy)carbonyl)amino)-2-(2,2,2-trideuteroethyl)-6-(trifluoromethyl)-3,4-dihydroquinoline-1(2H)-carboxylate (compound of Formula 1 wherein Y1a-c; Y3a-c; Y5a-c=D) is prepared according to the general method set forth in Example 50, starting from the product of Example 38.

### Example 53

N-Vinyl-carbamic acid benzylester. A 500 mL flask was charged with sodium azide (22.8 g, 0.35 mol), water (65 mL), toluene (65 mL), and Adogen 464 (0.03 g). The mixture was cooled with stirring in an ice-water bath and acryloylchloride (30.0 g, 0.33 mol) was added dropwise over a period of 2 h at 0-5° C. The mixture was stirred for a further 45 mins at 0-5° C. The organic phase was then separated and stored at 0-5° C. until used. A 500 mL flask was equipped with a temperature controller, distillation head, spiral condenser and 500 mL receiver flask. The distillation flask was charged with toluene (100 mL) and phenothiazine (0.2 g) and heated to 105-110° C. The receiver flask was charged with benzylalcohol (28.7 g), phenothiazine (0.02 g) and triethylamine (0.1 g) and the mixture was cooled in an ice-water bath with stirring. The solution of acryloylazide was added dropwise in to the distillation flask over a period of 3.5 h. [Note: acryloylazide solution was added in portions to dropping funnel in order to maintain the temperature as close to 0-5° C. as possible]. The distillate was collected into the benzylalcohol mixture with a head temperature of 80-100° C. An additional 100 mL (2×50 mL) was charged to the distillation flask during the distillation due to the volume becoming low. After complete addition of the acryloylazide solution a further 10 mL of toluene was distilled until a constant head temperature of 110° C. The receiver was isolated from the distillation set up and stirred at 0-5° C. for 2 h before being allowed to warm to room temperature overnight. The mixture was then concentrated in vacuo until a weight of 40-50 g. The residue was cooled in an ice bath to form a white solid which was slurried in hexane (100 mL) for 30 mins and filtered to give a white solid (17.2 g, 29%).

### Example 54

2,2,2-trideuteroethylchloroformate: Phosgene (20% solution in toluene, 20.5 ml, 41.25 mmol, 1 eq.) was charged to a 100 ml 3 necked flask and cooled to 0° C. Anhydrous pyridine (3.75 ml, 45.38 mmol, 1.1 eq.) was added dropwise maintaining the temperature at 0-5° C. The formation of a white precipitate occurred. 2,2,2-trideutero-ethanol (2.5 ml, 41.25 mmol, 1 eq.) was added dropwise allowing the mixture to warm to room temperature to aid mobility of the slurry. The mixture was allowed to stir at room temperature for 2 h and then used directly.

### Example 55

pentadeuteroethylchloroformate: The title compound was prepared from d5-ethanol according to the general method set forth in Example 54.

### Example 56

trideuteromethylchloroformate: The title compound was prepared from d3-methanol according to the general method set forth in Example 54.

### Example 57

3,3,3-trideuteropropionaldehyde. 3,3,3-trideuteropropanol (20 g) was charged to a 500 ml 3 necked round bottomed flask fitted with a dropping funnel and a condenser which was connected to a Huber at 60° C. A condenser set for downward distillation was connected to the top of the first condenser and to a 100 ml receiver flask cooled in an ice bath. The 3,3,3-trideuteropropanol was heated to reflux (97° C.) with stirring and a solution of K₂Cr₃O₇ (32.8 g) in water (200 ml) and conc. H₂SO₄ (24 ml) was added dropwise over 30 min. The pot temperature dropped to 86° C. then remained constant throughout addition. The head temperature was 33-37° C. throughout the addition. After complete addition of the reagents the mixture was heated until the head temperature dropped below 30° C. The desired aldehyde (6.4 g) was collected in 33% yield. ¹H NMR corresponded to the desired product.

### Example 58

cis-ethyl 4-(3,5-bis(trifluoromethyl)benzylamino)-2-(2,2,2-trideuteroethyl)-6-(trifluoromethyl)-3,4-dihydroquinoline-1(2H)-carboxylate (Compound (O)₁₋₄ wherein Y3a-c=D). The product from example 17 was reacted according to the procedure in Example 33 to provide the title compound in 98% yield.

### Example 59

cis-ethyl 4-(3,5-bis(trifluoromethyl)benzylamino)-2-pentadeuteroethyl-6-(trifluoromethyl)-3,4-dihydroquinoline-1(2H)-carboxylate (Compound (±)₁₋₄ wherein Y3a-c and Y4a-b=D). The product from example 18 was reacted according to the procedure in Example 33 to provide the title compound in 93% yield and a purity of >95% by ¹H NMR.

### Example 60

cis-2,2,2-trideuteroethyl 4-(3,5-bis(trifluoromethyl)benzylamino)-2-ethyl-6-(trifluoromethyl)-3,4-dihydroquinoline-1(2H)-carboxylate (Compound (±) 14 wherein Y1a-c=D). The product from example 19 was reacted according to the procedure in Example 33 to provide the title compound in 61% yield.

### Example 61

cis-pentadeuteroethyl 4-(3,5-bis(trifluoromethyl)benzylamino)-2-ethyl-6-(trifluoromethyl)-3,4-dihydroquinoline-1(2H)-carboxylate (Compound (±) 14 wherein Y1a-c and Y2a-b=D). The product from example 20 was reacted according to the procedure in Example 33 to provide the title compound in 77% yield.

### Example 62

cis-2,2,2-trideuteroethyl 4-(3,5-bis(trifluoromethyl)benzylamino)-2-(2,2,2-trideutero)-ethyl-6-(trifluoromethyl)-3,4-dihydroquinoline-1(2H)-carboxylate (Compound (±)₁₋₄ wherein Y1a-c and Y3a-c=D). The product from example 21 was reacted according to the procedure in Example 33 to provide the title compound in 100% yield.

### Example 63

cis-2,2,2-trideuteroethyl 4-(3,5-bis(trifluoromethyl)benzylamino)-2-pentadeuteroethyl-6-(trifluoromethyl)-3,4-dihydroquinoline-1(2H)-carboxylate (Compound (±)₁₋₄ wherein Y1a-c; Y3a-c; and Y4a-b=D). The product from example 22 was reacted according to the procedure in Example 33 to provide the title compound in 80% yield with purity >95% by ¹H NMR.

### Example 64

cis-pentadeuteroethyl 4-(3,5-bis(trifluoromethyl)benzylamino)-2-(2,2,2-trideuteroethyl)-6-(trifluoromethyl)-3,4-dihydroquinoline-1(2H)-carboxylate (Compound (±)₁₋₄ wherein Y1a-c; Y2a-b; and Y3a-c=D). The product from example 23 was reacted according to the procedure in Example 33 to provide the title compound in 85% yield and with a purity of >95% by ¹H NMR.

### Example 65

cis-pentadeuteroethyl 4-(3,5-bis(trifluoromethyl)benzylamino)-2-pentadeuteroethyl-6-(trifluoromethyl)-3,4-dihydroquinoline-1(2H)-carboxylate (Compound (±)₁₋₄ wherein Y1a-c; Y2a-b; Y3a-c; and Y4a-b=D). The product from example 24 was reacted according to the procedure in Example 33 to provide the title compound in 59% yield with >95% purity by ¹H NMR.

### Example 66

cis-ethyl 4-((3,5-bis(trifluoromethyl)benzyl)-(methoxycarbonyl)amino)-2-ethyl-6-(trifluoromethyl)-3,4-dihydroquinoline-1(2H)-carboxylate ((±) compound of Formula 1 wherein all Y═H). To a stirred solution of the product from Example 33 (10.0 g, 18.4 mmol, 1 equiv.) in THF (100 ml) under a N₂ atmosphere was added Na₂CO₃ (9.8 g, 92.2 mmol, 5 equiv.). Methyl chloroformate (8.7 g, 92.2 mmol, 5 equiv.) was added dropwise. The reaction mixture was stirred for a period of 48 h resulting in complete consumption of the starting material as indicated by LC analysis. The reaction mixture was concentrated to ˜50 ml. EtOAc (250 ml) was added and the organic layer was washed with 1M HCl (100 ml), sat. aq. NaHCO₃ (100 ml) and brine (100 ml). The organic layer was dried over MgSO₄ and concentrated in vacuo to give a colorless oil. The oil was dissolved in abs. EtOH (33 ml) and stirred overnight to form a white precipitate. The solid was filtered and dried under vacuum to yield the title product: 8.2 g, 74% yield in a purity of >95% by ¹H NMR.

### Example 67

cis-ethyl 4-((3,5-bis(trifluoromethyl)benzyl)(trideutero-methoxycarbonyl)amino)-2-ethyl-6-(trifluoromethyl)-3,4-dihydroquinoline-1(2H)-carboxylate ((±) compound of Formula 1 wherein Y5a-c=D). The product from example 33 was reacted with the product from example 56 according to the procedure in Example 66 to provide the title compound in 40% yield in a purity of >95% by ¹H NMR.

### Example 68

cis-pentadeuteroethyl 4-((3,5-bis(trifluoromethyl)benzyl)-((trideuteromethoxy)carbonyl)amino)-2-ethyl)-6-(trifluoromethyl)-3,4-dihydroquinoline-1(2H)-carboxylate ((i) compound of Formula 1 wherein Y1a-c; Y2a-b; and Y5a-c=D). The product from example 61 was reacted with the product from example 56 according to the procedure in Example 66 to provide the title compound in 19% yield in a purity of >95% by ¹H NMR.

### Example 69

cis-pentadeuteroethyl 4-((3,5-bis(trifluoromethyl)benzyl)-((trideuteromethoxy)carbonyl)amino)-2-(2,2,2-trideuteroethyl)-6-(trifluoromethyl)-3,4-dihydroquinoline-1(2H)-carboxylate ((±) compound of Formula 1 wherein Y1a-c; Y2a-b; Y3a-c; and Y5a-c=D). The product from example 64 was reacted with the product from example 56 according to the procedure in Example 66 to provide the title compound in 20% yield in a purity of >95% by ¹H NMR.

### Example 70

cis-pentadeuteroethyl 4-((3,5-bis(trifluoromethyl)benzyl)-((trideuteromethoxy)carbonyl)amino)-2-pentadeuteroethyl-6-(tri fluoromethyl)-3,4-dihydroquinoline-1(2H)-carboxylate ((i) compound of Formula 1 wherein Y1a-c; Y2a-b; Y3a-c; Y4a-b; and Y5a-c=D). The product from example 65 was reacted with the product from example 56 according to the procedure in Example 66 to provide the title compound in 23% yield in a purity of >95% by ¹H NMR.

### Example 71

cis-2,2,2-trideuteroethyl 4((3,5-bis(trifluoromethyl)benzyl)-(methoxycarbonyl)amino)-2-ethyl-6-(trifluoromethyl)-3,4-dihydroquinoline-1(2H)-carboxylate ((±)compound of Formula 1 wherein 1a-c=D). The product from example 60 was reacted according to the procedure in Example 66 to provide the title compound in 62% yield in a purity of >95% by ¹H NMR.

### Example 72

cis-ethyl 4-((3,5-bis(trifluoromethyl)benzyl)-(methoxycarbonyl)amino)-2-(2,2,2-trideuteroethyl)-6-(trifluoromethyl)-3,4-dihydroquinoline-1(2H)-carboxylate (compound of Formula 1 wherein Y3a-c=D). The product from example 58 was reacted according to the procedure in Example 66 to provide the title compound in 45% yield in a purity of >95% by ¹H NMR.

### Example 73

cis-ethyl 4-((3,5-bis(trifluoromethyl)benzyl)-(methoxycarbonyl)amino)-2-pentadeuteroethyl-6-(trifluoromethyl)-3,4-dihydroquinoline-1(2H)-carboxylate (compound of Formula 1 wherein Y3a-c and Y4a-b=D). The product from example 59 was reacted according to the procedure in Example 66 to provide the title compound in 48% yield in a purity of >95% by ¹H NMR.

### Example 74

cis-2,2,2-trideuteroethyl 4-((3,5-bis(trifluoromethyl)benzyl)-(methoxycarbonyl)amino)-2-(2,2,2-trideuteroethyl)-6-(trifluoromethyl)-3,4-dihydroquinoline-1(2H)-carboxylate (compound of formula 1 wherein Y1a-c and Y3a-c=D). The product from example 62 was reacted according to the procedure in Example 66 to provide the title compound in 45% yield in a purity of >95% by ¹H NMR.

### Example 75

cis-pentadeuteroethyl 4((3,5-bis(trifluoromethyl)benzyl)-(methoxycarbonyl)amino)-2-(2,2,2-trideuteroethyl)-6-(trifluoromethyl)-3,4-dihydroquinoline-1(2H)-carboxylate (compound of formula 1 wherein Y1a-c; Y2a-b; and Y3a-c=D). The product from example 64 was reacted according to the procedure in Example 66 to provide the title compound in 42% yield in a purity of >95% by ¹H NMR.

### Example 76

cis-pentadeuteroethyl 4-((3,5-bis(trifluoromethyl)benzyl)-(methoxycarbonyl)amino)-2-ethyl-6-(trifluoromethyl)-3,4-dihydroquinoline-1(2H)-carboxylate (compound of Formula 1 wherein Y1a-c and Y2a-b=D). The product from example 61 was reacted according to the procedure in Example 66 to provide the title compound in 52% yield in a purity of >95% by ¹H NMR.

### Example 77

cis-pentadeuteroethyl 4-((3,5-bis(trifluoromethyl)benzyl)-(methoxycarbonyl)amino)-2-pentadeuteroethyl-6-(trifluoromethyl)-3,4-dihydroquinoline-1(2H)-carboxylate (compound of Formula 1 wherein Y1a-c; Y2a-b; Y3a-c; and Y4a-b=D). The product from example 65 was reacted according to the procedure in Example 66 to provide the title compound in 44% yield in a purity of >95% by ¹H NMR.

### Example 78

cis-2,2,2-trideuteroethyl 4-((3,5-bis(trifluoromethyl)benzyl)-(methoxycarbonyl)amino)-2-pentadeuteroethyl-6-(trifluoromethyl)-3,4-dihydroquinoline-1(2H)-carboxylate (compound of Formula 1 wherein Y1a-c; Y3a-c; and Y4a-b=D). The product from example 63 was reacted according to the procedure in Example 66 to provide the title compound in 63% yield in a purity of >95% by ¹H NMR.

### Example 79

Microsomal Assay: Certain in vitro liver metabolism studies have been described previously in the following references, each of which is incorporated herein in their entirety: Obach, R. S. Drug Metab Disp 1999, 27, p. 1350 “Prediction of human clearance of twenty-nine drugs from hepatic microsomal intrinsic clearance data: An examination of in vitro half-life approach and nonspecific binding to microsomes”; Houston, J. B. et al., Drug Metab Rev 1997, 29, p. 891 “Prediction of hepatic clearance from microsomes, hepatocytes, and liver slices”; Houston, J. B. Biochem Pharmacol 1994, 47, p. 1469 “Utility of in vitro drug metabolism data in predicting in vivo metabolic clearance”; Iwatsubo, T et al., Pharmacol Ther 1997, 73, p. 147 “Prediction of in vivo drug metabolism in the human liver from in vitro metabolism data”; and Lave, T. et al., Pharm Res 1997, 14, p. 152 “The use of human hepatocytes to select compounds based on their expected hepatic extraction ratios in humans”.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., Absorption Systems L. P. (Exton, Pa.)). The incubation mixtures are prepared as follows:

Reaction Mixture Composition

| | | |
|---|---|---|
| Liver Microsomes | 1.0 | mg/mL |
| NADPH | 1 | mM |
| Potassium Phosphate, pH 7.4 | 100 | mM |
| Magnesium Chloride | 10 | mM |
| Test Compound | 1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (not the test compound). The reaction is initiated by the addition of cofactors (not into the negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as Compound 1, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

The following compounds were tested in the human microsome assay: Compound 1, Compound 101, Compound 102, Compound 103, Compound 104, Compound 105, Compound 106, Compound 107, Compound 108, Compound 109, Compound 110, Compound 112 and Compound 113.

Deuteration was shown to affect the stability of the compounds in the human microsome assay. Notably, compounds having deuteration of the ethyl carbamate portion exhibited significantly greater stability at 60 minutes compared to the parent compound in the human microsome assay.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Another embodiment is a compound of any of the formulae herein made by a process delineated herein, including the processes exemplified in the schemes and examples herein. Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a disease, disorder or symptom thereof delineated herein.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of Formula 1: 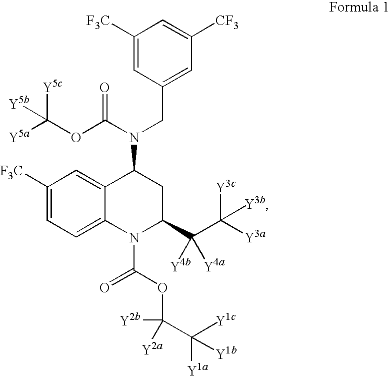
wherein Y^{1c}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c} are each independently selected from the group consisting of hydrogen, deuterium, or fluorine; and wherein at least one Y is deuterium;
or a pharmaceutically acceptable salt, thereof.

2. The compound according to claim 1, wherein Y^{1a}, Y^{1b}, and Y^{1c} are each deuterium.

3. The compound according to claim 2, wherein Y^{1a}, Y^{1b}, Y^{1c}, Y^{2a} and Y^{2b} are each deuterium.

4. The compound according to claim 1, wherein Y^{3a}, Y^{3b}, and Y^{3c} are each deuterium.

5. The compound according to claim 1, wherein at least five substituents selected from the group consisting of Y^{1a}, Y^{1b}, Y^{1c}, Y^{2a}, Y^{2b}, Y^{3a}, Y^{3b}, Y^{3c}, Y^{4a}, and Y^{4b} are deuterium.

6. The compound according to claim 1, wherein at least five substituents selected from the group consisting of Y^{1c}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c} are deuterium.

7. The compound according to claim 5, wherein the compound comprises at least six deuterium atoms and wherein at least one substituent selected from the group consisting of Y^{1a}, Y^{1b}, Y^{1c}, Y^{2a}, Y^{2b}, Y^{3a}, Y^{3b}, Y^{3c}, Y^{4a}, and Y^{4b} is deuterium.

8. The compound according to claim 7, wherein at least six substituents selected from the group consisting of Y^{1c}, Y^{1b}, Y^{1c}, Y^{2a}, Y^{2b}, Y^{3a}, Y^{3b}, Y^{3c}, Y^{4a}, Y^{4b}, Y^{5a}, Y^{5b}, and Y^{5c} are deuterium.

9-16. (canceled)

17. A compound selected from the group consisting of: (2S,4S)-ethyl 4-((3,5-bis(trifluoromethyl)benzyl)(methoxycarbonyl)amino)-2-(2,2,2-trideuteroethyl)-6-(trifluoromethyl)-3,4-dihydroquinoline-1 (2H-carboxylate; (2S,4S)-ethyl 4-((3,5-bis(trifluoromethyl)benzyl)(methoxycarbonyl)amino)-2-pentadeuteroethyl-6-(trifluoromethyl)-3,4-dihydroquinoline-1(2H-carboxylate; (2S,4S)-2,2,2-trideuteroethyl 4((3,5-bis(trifluoromethyl)benzyl)(methoxycarbonyl)amino)-2-ethyl-6-(trifluoromethyl)-3,4-dihydroquinoline-1(2H-carboxylate; (2S,4S)-pentadeuteroethyl 4-((3,5-bis(trifluoromethyl)benzyl)(methoxycarbonyl)amino)-2-ethyl-6-(trifluoromethyl)-3,4-dihydroquinoline-1(2H-carboxylate; (2S,4S)-2,2,2-trideuteroethyl 4-((3,5-bis(trifluoromethyl)benzyl)(methoxycarbonyl)amino)-(2,2,2-trideutero)-ethyl-6-(trifluoromethyl)-3,4-dihydroquinoline-1(2H-carboxylate; (2S,4S)-2,2,2-trideuteroethyl 4-((3,5-bis(trifluoromethyl)benzyl)(methoxycarbonyl)amino)-2-pentadeuteroethyl-6-(trifluoromethyl)-3,4-dihydroquinoline-1(2H-carboxylate; (2S,4S)-pentadeuteroethyl 4((3,5-bis(trifluoromethyl)benzyl)(methoxycarbonyl)amino)-2-(2,2,2-trideuteroethyl)-6-(trifluoromethyl)-3,4-dihydroquinoline-1(2H-carboxylate; (2S,4S)-pentadeuteroethyl 4-((3,5-bis(trifluoromethyl)benzyl)(methoxycarbonyl)amino)-2 pentadeuteroethyl-6-(trifluoromethyl)-3,4-dihydroquinoline-1(2″-carboxylate, or an isotopoloque thereof or a salt thereof.

18-23. (canceled)

24. A pharmaceutical composition comprising an effective amount of a compound of claim 1; or a pharmaceutically acceptable salt thereof; and an acceptable carrier.

25-30. (canceled)

31. The composition according to claim 24, further comprising an effective amount of a second therapeutic agent, wherein said second therapeutic agent is useful for treating or preventing a condition in a patient selected from coronary heart disease including myocardial infarction, atherosclerosis, peripheral vascular disease, cardiovascular disorders, angina, ischemia, cardiac ischemia, stroke, reperfusion injury, angioplastic restenosis, hypertension, diabetes, including its vascular complications, endotoxemia, and intermittent claudication.

32. The composition according to claim 31, wherein said second therapeutic agent is selected from an HMG-CoA reductase inhibitor, a glucosidase and/or amylase inhibitor, and a growth hormone secretagogue; or a pharmaceutically acceptable salt thereof.

33. (canceled)

34. A method of inhibiting CETP receptor in a patient comprising the step of administering to said patient a composition comprising an effective amount of a compound of claim 1, or a pharmaceutically acceptable salt thereof; and an acceptable carrier.

35. A method of treating a subject suffering from or susceptible to atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension; vascular complications of diabetes, obesity or endotoxemia, said method comprising the step of administering to said subject a composition comprising an effective amount of a compound of claim 1, or a pharmaceutically acceptable salt thereof; and an acceptable carrier.

36-49. (canceled)

50. The method according to claim 35, comprising the additional step of administering to said patient a second therapeutic agent, wherein said second therapeutic agent is conventionally used for treating or preventing a condition selected from coronary heart disease including myocardial infarction, atherosclerosis, peripheral vascular disease, cardiovascular disorders, angina, ischemia, cardiac ischemia, stroke, reperfusion injury, angioplastic restenosis, hypertension, diabetes, including its vascular complications, endotoxemia, and intermittent claudication.

51. The method according to claim 50, wherein said second therapeutic agent is selected from one or more of an HMG-CoA reductase inhibitor, a glucosidase and/or amylase inhibitor, and a growth hormone secretagogue; or a pharmaceutically acceptable salt thereof.

52-64. (canceled)

65. A compound of formula 2: 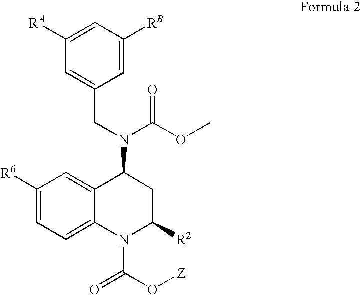
wherein:
R^{A} and R^{B} are each independently trifluoromethyl, deuteromethyl or chloro;
R² is ethyl, isopropyl, cyclopropyl, cyclobutyl, or methoxymethyl;
R⁶ is trifluoromethyl, deuteromethyl or chloro;
Z is ethyl, propyl, isopropyl, tert-butyl, or 2-hydroxyethyl, each optionally substituted with from 1-9 independent substituents, each selected from fluorine and hydroxyl groups;
wherein one or more H atoms in Z or R² is replaced by deuterium;
or a salt thereof.
